(19) United States
(12) Patent Application Publication
(10) Pub. No.: US 2025/0172483 A1
(43) Pub. Date: May 29, 2025

(54) SINGLE SIDE MAGNETIC PARTICLE IMAGING DEVICE

(71) Applicant: GIST(Gwangju Institute of Science and Technology), Gwangju (KR)

(72) Inventors: Jung Won YOON, Gwangju (KR); Luu Thanh CAO, Gwangju (KR); Seung Jun OH, Gwangju (KR)

(73) Assignee: GIST(Gwangju Institute of Science and Technology), Gwangju (KR)

(21) Appl. No.: 18/909,335

(22) Filed: Oct. 8, 2024

(30) Foreign Application Priority Data
Nov. 27, 2023 (KR) 10-2023-0166788

(51) Int. Cl. G01N 15/1031; A61B 5/0515

(52) U.S. Cl. CPC G01N 15/1031; A61B 5/0515

(57) ABSTRACT

The present disclosure relates to a device that can image magnetic particles through a Field Free Line (FFL) formed in a single direction. The single side magnetic particle imaging device according to an embodiment of the present disclosure includes: a pair of selection coils arranged in parallel in an x-axial direction and configured to create a y-axial Field Free Line (FFL) in a Field of View (FOV); a driver coil disposed under the pair of selection coils and configured to move the FFL in a z-axial direction by generating a magnetic field; An excitation coil disposed over the pair of selection coils and configured to excite magnetic fields in the FOV by generating a magnetic field in the FOV; and a pair of receiver coils arranged in parallel in a y-axial direction over the excitation coil and configured to receive signals generated by the magnetic particles, respectively.

### CROSS REFERENCE TO RELATED APPLICATION

The present application claims priority to Korean Patent Applications No. 10-2023-0166788, filed Nov. 27, 2023, the entire contents of which are incorporated herein for all purposes by this reference.

### BACKGROUND

### Technical Field

The present disclosure relates to a device that can image magnetic particles through a Field Free Line (FFL) formed in a single direction.

### Description of the Related Art

A Magnetic Particle Imaging (MPI) device has a limitation that it cannot obtain an anatomical image in comparison to a magnetic resonance imaging device or an X-ray device.

### SUMMARY

An objective of the present disclosure is to provide a portable small-size magnetic particle imaging device that can image magnetic particles through a Field Free Line (FFL) formed in a single direction.

The objectives of the present disclosure are not limited to those described above and other objectives and advantages not stated herein may be understood through the following description and may be clear by embodiments of the present disclosure. Further, it would be easily known that the objectives and advantages of the present disclosure may be achieved by the configurations described in claims and combinations thereof.

In order to achieve the objectives, a single side magnetic particle imaging device according to an embodiment of the present disclosure includes: a pair of selection coils arranged in parallel in an x-axial direction and configured to create a y-axial Field Free Line (FFL) in a Field of View (FOV); a driver coil disposed under the pair of selection coils and configured to move the FFL in a z-axial direction by generating a magnetic field; An excitation coil disposed over the pair of selection coils and configured to excite magnetic fields in the FOV by generating a magnetic field in the FOV; and a pair of receiver coils arranged in parallel in a y-axial direction over the excitation coil and configured to receive signals generated by the magnetic particles, respectively.

The single side magnetic particle imaging device of the present disclosure is a small-sized coil-based device and has the advantage that it can image an object while being carried anywhere and can secure a wide Field of View (FOV) in comparison to permanent magnet-based devices by imaging magnetic particles through an FFL that is formed in a single direction.

Detailed effects of the present disclosure in addition to the above effects will be described with the following detailed description for accomplishing the present disclosure.

### BRIEF DESCRIPTION OF THE DRAWINGS

The above and other objectives, features and other advantages of the present disclosure will be more clearly understood from the following detailed description when taken in conjunction with the accompanying drawings, in which:

FIG. 1 is an exploded perspective view of a single side magnetic particle imaging device according to an embodiment of the present disclosure;

FIG. 2 is a view showing the structure of the single side magnetic particle imaging device according to an embodiment of the present disclosure;

FIG. 3A to FIG. 3C are views illustrating an FFL that is created by a pair of selection coils according to an embodiment of the present disclosure;

FIG. 4A and FIG. 4B are views illustrating transverse movement of an FFL;

FIG. 5A and FIG. 5B are views showing a driver coil disposed under a pair of selection coils;

FIG. 6 is a view illustrating longitudinal movement of an FFL;

FIG. 7 is a view showing an excitation coil disposed over a pair of selection coils;

FIG. 8 is a view showing a magnetic field that is formed in an FOV by an excitation coil; and

FIG. 9 is a view showing a pair of receiver coils disposed over an excitation coil.

### DETAILED DESCRIPTION

The objects, characteristics, and advantages will be described in detail below with reference to the accompanying drawings, so those skilled in the art may easily achieve the spirit of the present disclosure. However, in describing the present disclosure, detailed descriptions of well-known technologies will be omitted so as not to obscure the description of the present disclosure with unnecessary details. Hereinafter, exemplary embodiments of the present disclosure will be described with reference to accompanying drawings. The same reference numerals are used to indicate the same or similar components in the drawings.

Although terms “first”, “second”, etc. are used to describe various components in the specification, it should be noted that these components are not limited by the terms. These terms are used to discriminate one component from another component and it is apparent that a first component may be a second component unless specifically stated otherwise.

Further, when a certain configuration is disposed “over (or under)” or “on (beneath)” a component in the specification, it may mean not only that the certain configuration is disposed on the top (or bottom) of the component, but that another configuration may be interposed between the component and the certain configuration disposed on (or beneath) the component.

Further, when a certain component is “connected”, “coupled”, or “jointed” to another component in the specification, it should be understood that the components may be directly connected or jointed to each other, but another component may be “interposed” between the components or the components may be “connected”, “coupled”, or “jointed” through another component.

Further, singular forms that are used in this specification are intended to include plural forms unless the context clearly indicates otherwise. In the specification, terms “configured”, “include”, or the like should not be construed as necessarily including several components or several steps described herein, in which some of the components or steps may not be included or additional components or steps may be further included.

Further, the term “A and/or B” stated in the specification means that A, B, or A and B unless specifically stated otherwise, and the term “C to D” means that C or more and D or less unless specifically stated otherwise.

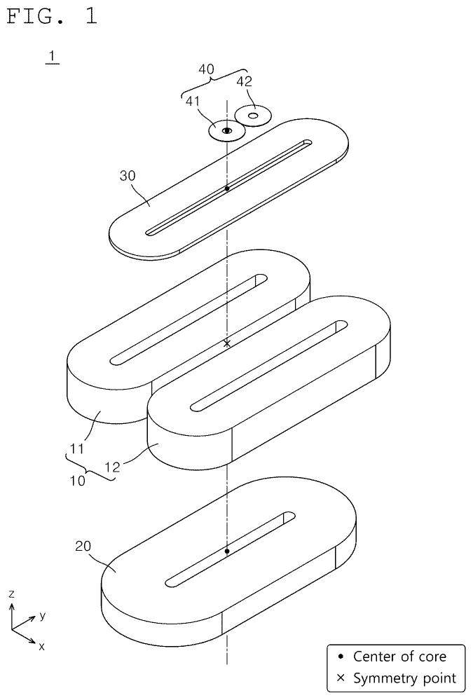

The present disclosure relates to a device that can image magnetic particles through a Field Free Line (FFL) formed in a single direction. Hereafter, a single side magnetic particle imaging device according to an embodiment of the present disclosure is described in detail with reference to FIGS. 1 to 9.

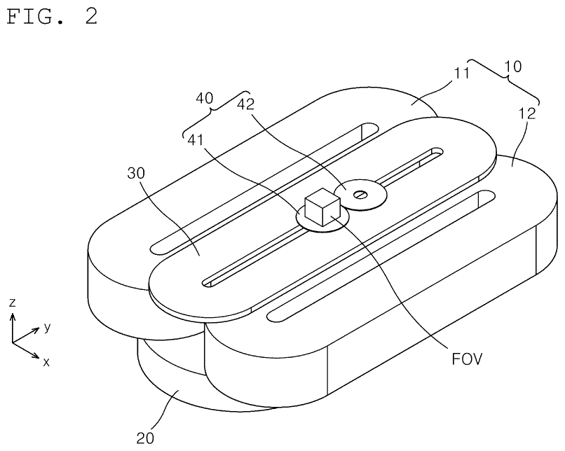

FIG. 1 is an exploded perspective view of a single side magnetic particle imaging device according to an embodiment of the present disclosure and FIG. 2 is a view showing the structure of the single side magnetic particle imaging device according to an embodiment of the present disclosure.

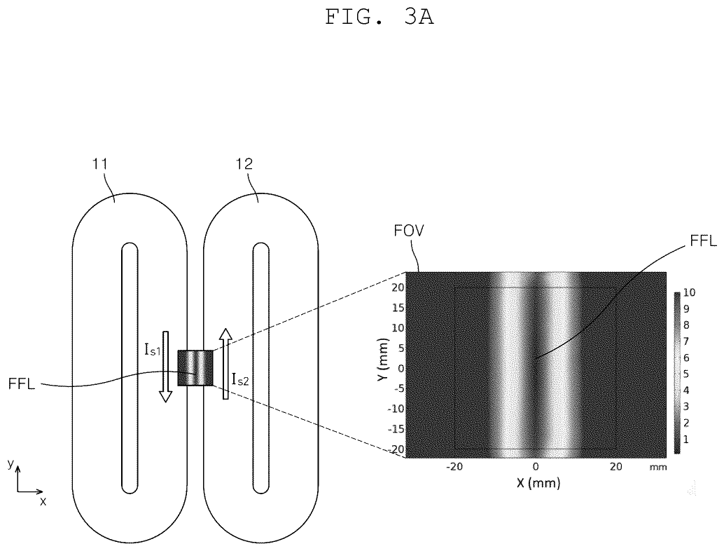

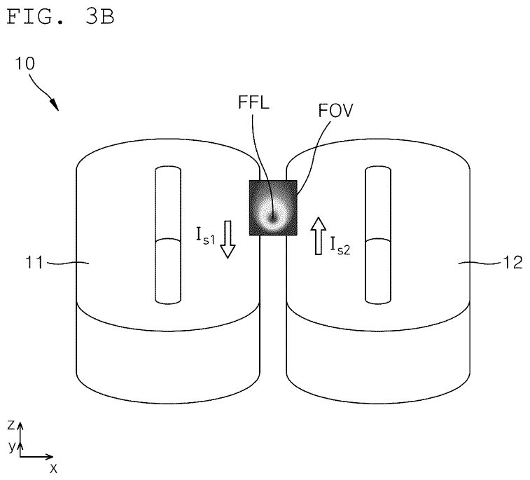

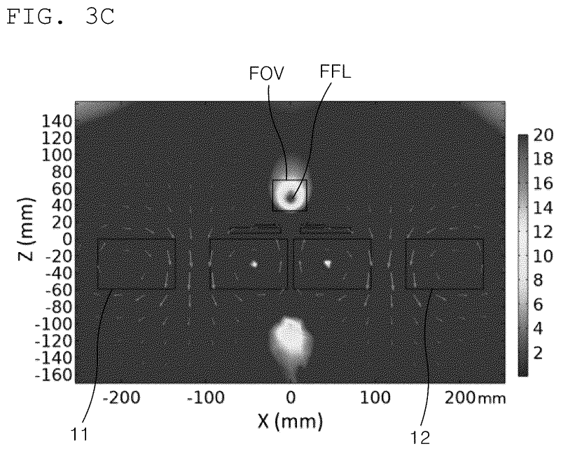

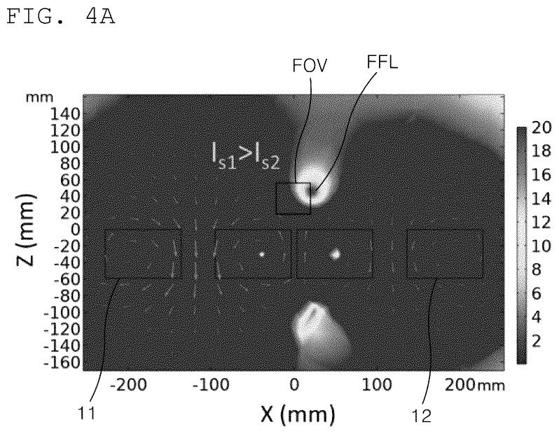

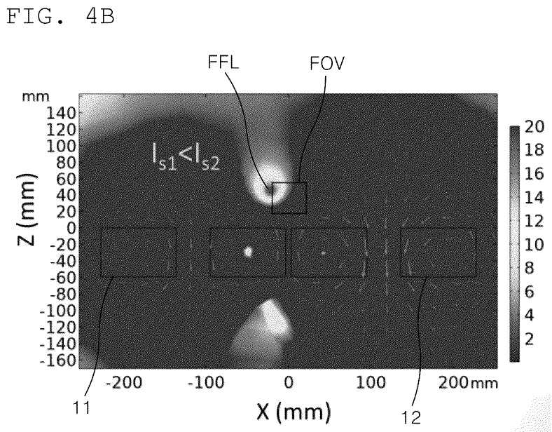

FIG. 3A to FIG. 3C are views illustrating an FFL that is created by a pair of selection coils according to an embodiment of the present disclosure and FIG. 4A and FIG. 4B are views illustrating transverse movement of an FFL.

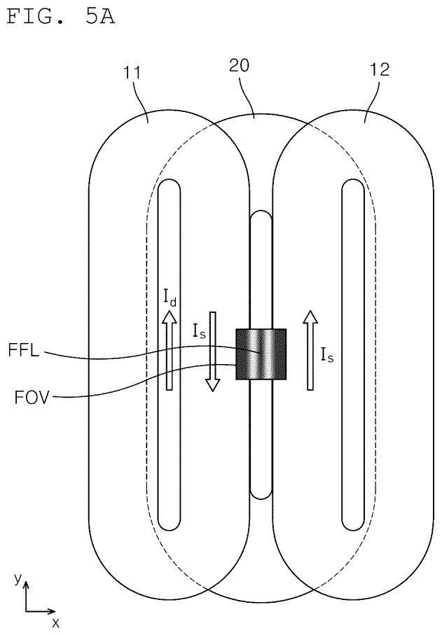

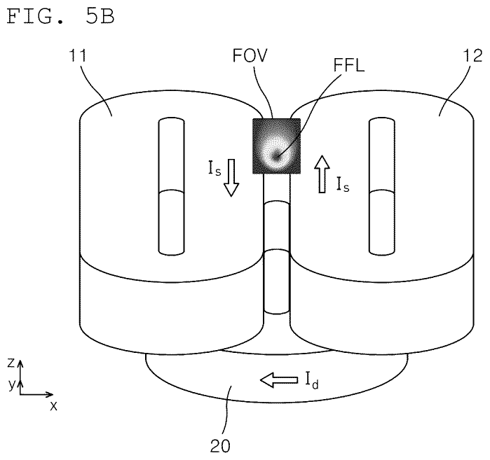

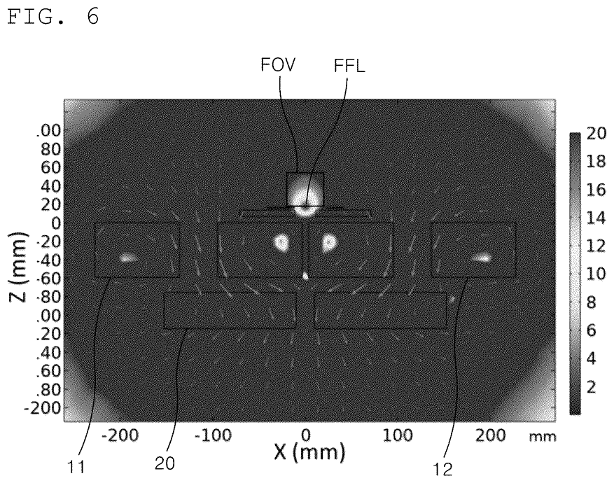

FIG. 5A and FIG. 5B are views showing a driver coil disposed under a pair of selection coils and FIG. 6 is a view illustrating longitudinal movement of an FFL.

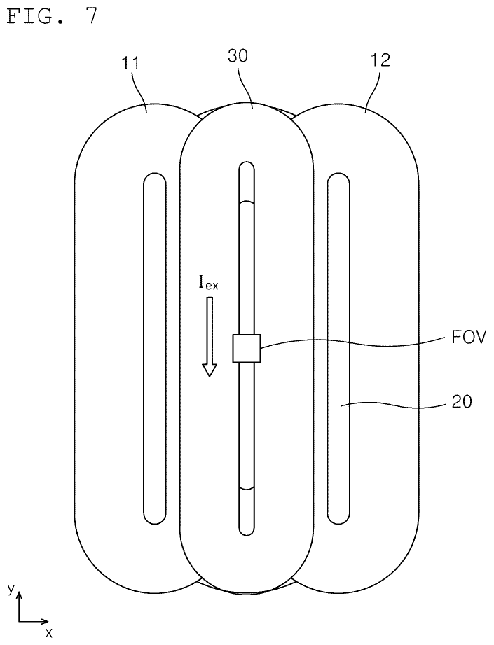

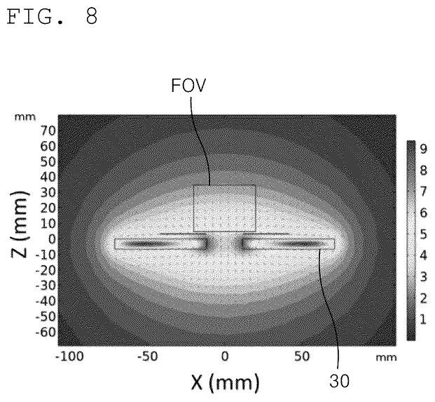

FIG. 7 is a view showing an excitation coil disposed over a pair of selection coils and FIG. 8 is a view showing a magnetic field that is formed in an FOV by an excitation coil.

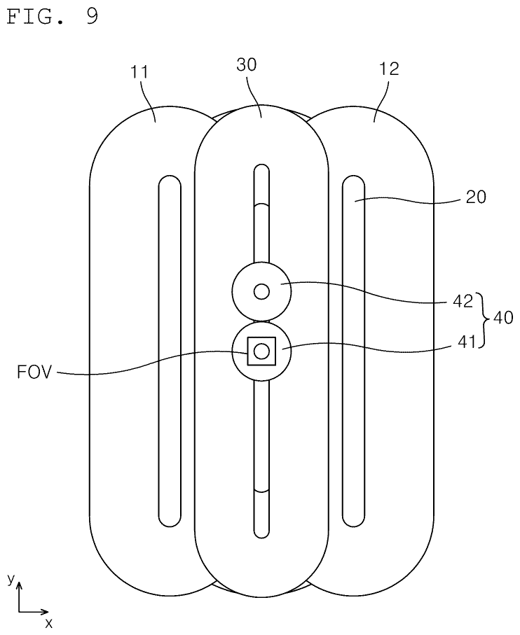

FIG. 9 is a view showing a pair of receiver coils disposed over an excitation coil.

Referring to FIG. 1 and FIG. 2, a single side magnetic particle imaging device 1 according to an embodiment of the present disclosure may include a pair of selection coils 10, a driver coil 20, an excitation coil 30, and a pair of receiver coils 40, and, though not shown in the figures, may include a processor that controls the amount of current that is applied to each of the coils 10, 20, and 30 and performs an imaging process on magnetic particles on the basis of a signal acquired from the pair of receiver coils 40.

However, the a single side magnetic particle imaging device 1 shown in FIG. 1 and FIG. 2 is based on an embodiment, the components thereof are not limited to the embodiment shown in FIG. 1 and FIG. 2, and if necessary, some components may be added, changed, or removed.

The single side magnetic particle imaging device 1 of the present disclosure, basically, can detect a harmonic signal according to a non-linear characteristic in which magnetic particles have in a gradient magnetic field and obtain an image on the basis of the harmonic signal.

To this end, the single side magnetic particle imaging device 1 of the present disclosure can create a Field Free Line (FFL) (hereafter, FFL) in a Field of View (FOV) (hereafter, FOV), and in detail, can create an FFL using the pair of selection coils 10.

The single side magnetic particle imaging device 1 of the present disclosure has a length less than 10 cm, so the device 1 can be used in the type of coming in direct contact with the body (e.g., the skin) of a patient to specify a diseased part such as cancer.

3-dimensional arrangement of the components shown in FIG. 1 and FIG. 2 is described on the basis of an x-axis, a y-axis, and a z-axis that are perpendicular to each other, and the +x-axial direction is described as the right, the −x-axial direction is described as the left, the +z-axial direction is described as up, and the −z-axial direction is described as down, but this is for the convenience of description and it is apparent that any one axis may be changed to and understood as another axis.

Referring to FIG. 3A to FIG. 3C, the pair of selection coils 10 of the present disclosure may by arranged in parallel in the x-axial direction and may create an FFL extending in the y-axial direction in an FOV set over them. The selection coils 11 and 12 each may be an elliptical flat coil of which the major axis is parallel with the y-axial direction, and in the following direction, the pair of selection coils 10 is separately described as first and second selection coils 11 and 12, if necessary.

The pair of selection coils 10 can function as Maxell coils to form an FFL, and to this end, currents Iₛ₁ and Iₛ₂ of the same direction may be applied to the pair of selection coils 10. For example, clockwise currents Iₛ₁ and Iₛ₂ may flow in the pair of selection coils 10, respectively.

Referring to FIG. 3A again, since currents of the same direction flow in the pair of selection coils 10, currents of opposite direction can flow in the two adjacent surfaces of the coils 11 and 12, respectively. In more detail, the direction of the current Iₛ₁ flowing on the right side of the first selection oil 11 and the direction of the current Iₛ₂ flowing on the left side of the second selection coil 12 that are adjacent to each other may be opposite to each other.

The magnetic field generated at the first and second selection coils 11 and 12 by flow of the currents Iₛ₁ and Iₛ₂ can form a gradient in the FOV, and accordingly, an FFL of the y-axial direction can be formed in the FOV.

Meanwhile, in order for the single side magnetic particle imaging device 1 to create an 3D image by selectively receiving only magnetic signals that are generated by magnetic particles, it is required to be able to control the position of an FFL in three axial directions in an FOV, and the x-axial position of an FFL can be determined in accordance with the difference in intensity of the currents flowing in the two selection coils 11 and 12 in the present disclosure.

Referring to FIG. 4A, in the arrangement of the selection coils 11 and 12, when the magnitude of the first current Iₛ₁ flowing in the first selection coil 11 is larger than the magnitude of the second current Iₛ₂ flowing in the second selection coil 12, the intensity of the magnetic field generated at the first selection coil 11 is relatively larger than the intensity the magnetic field generated at the second selection coil 12, whereby the position of an FFL can be moved (pushed in the +x-axial direction.

On the contrary, referring to FIG. 4B, when the magnitude of the second current Iₛ₂ flowing in the second selection coil 12 is larger than the magnitude of the first current Isi flowing in the first selection coil 11, the intensity the magnetic field generated at the second selection coil 12 is relatively larger than the intensity of the magnetic field generated at the first selection coil 11, whereby the position of an FFL can be moved (pushed in the −x-axial direction.

The x-axial displacement of an FFL is proportionate to the difference in intensity of the currents Iₛ₁ and Iₛ₂ flowing in the two selection coils 11 and 12, so it is possible to move the position of an FFL in the x-axial direction in an FOV by differently controlling the currents Iₛ₁ and Iₛ₂ flowing in the two selection coils 11 and 12.

Referring to FIG. 5A and FIG. 5B, the driver coil 20 of the present disclosure is disposed under the pair of selection coils 10 and can generate a magnetic field.

The driver coil 20 may be an elliptical flat coil of which the major axis is parallel with the y-axial direction and the center of the core thereof may be positioned under the center of the pair of selection coils 10. That is, as shown in FIG. 1, the center (center of core) of the driver coil 20 may be disposed on the same z-axis as the center of the pair of selection coils 10, in more detail, a symmetric point of the pair of selection coils 10.

The driver coil 20 can move an FFL in the z-axial direction by generating a magnetic field.

Referring to FIG. 5B and FIG. 6B, when a clockwise current I_{d} flows in the driver coil 20, a −z-axial magnetic field can be generated around an FFL, and accordingly, the position of the existing FFL can be moved (pushed) in the −z-axial direction.

On the contrary, though not shown in the figures, when a counterclockwise current I_{d} flows in the driver coil 20, a +z-axial magnetic field can be generated around an FFL, and accordingly, the position of the existing FFL can be moved (pushed) in the +z-axial direction.

The z-axial displacement of N FFL is proportioned to the intensity of a magnetic field generated at the driver coil 20 and the processor can move the position of an FFL in the z-axial direction in an FOV by controlling the amount of current flowing in the driver coil 20.

Referring to FIG. 7, the excitation coil 30 is disposed over the pair of selection coils 10 and can generate a magnetic field.

The excitation coil 30 may be an elliptical flat coil of which the major axis is parallel with the y-axial direction and the center (center of the core) thereof may be positioned over the center of the pair of selection coils 10. That is, as shown in FIG. 1, the center of core of the excitation coil 30 may be disposed on the same z-axis as the center of the pair of selection coils 10, in more detail, a symmetric point of the pair of selection coils 10.

When a magnetic field is formed in an FOV by the pair of selection coils 10 and the driver coil 20 described above, the excitation coil 30 can excite magnetic particles in the FOV by generating an additional magnetic field in the FOV.

In detail, the excitation coil 30 can excite magnetic fields by mixing a high-frequency magnetic field with a magnetic field formed in an FOV. In this case, the frequency of the magnetic field generated at the excitation coil 30 may be higher than the frequency of the magnetic field generated at the driver coil 20, and the intensity of the magnetic field generated at the excitation coil 30 may be much lower than the intensity of the magnetic field generated at the driver coil 20.

Referring to FIG. 8 separating a transverse cross-section of the excitation coil 30, when a counterclockwise current Iₑₓ flows in the excitation coil, the high-frequency magnetic field generated at the excitation coil 30 may be very low in intensity relatively to the magnetic field in the FOV shown in FIG. 6 and the frequency thereof may be higher than that. Accordingly, magnetic particles in an FOV can be excited by a high-frequency magnetic field without the position of an FFL influenced by the high-frequency magnetic field.

Referring to FIG. 1, FIG. 2, and FIG. 9, the pair of receiver coils 40 of the present disclosure may be circular flat coils arranged in parallel in the y-axial direction over the excitation coil 30. In this case, the pair of receiver coils 40 may be disposed to be directly adjacent to an FOV under the FOV.

The pair of receiver coils 40 each can receive a signal (hereafter, magnetic signal) that is generated by magnetic particles. In detail, the pair of receiver coils 40 converts a nonlinear signal that is generated by magnetization of magnetic particles in a mixed magnetic field of signals that are linearly received into induced electromotive force, thereby being able the sense a corresponding signal.

Meanwhile, in the present disclosure, a magnetic particle may be a superparamagnetic substance (e.g., a superparamagnetic nanoparticle) that generates a nonlinear signal when it is excited in a mixed magnetic field, and when the present disclosure is used to detect a carcinoma cell, the magnetic particle may include a receptor that bonds to a carcinoma cell.

The pair of coils 40 may be composed of a sensing coil 41 and an offset coil 42 to selectively receive only magnetic signals that are generated by magnetic particles except for the magnetic field mixed in an FOV. In this case, the sensing coil 41 and the offset coil 42 may be connected to each other and may have the same structure and the same number of winding, and the winding direction may be opposite to each other. For example, the sensing coil 41 and the offset coil 42 may be configured at one same conducting wire, and when the sensing coil 41 is wound clockwise, the offset coil 42 may be wound counterclockwise.

The magnetic fields generated at the driver coil 20 and the excitation coil 30 can induce electromotive forces having the same magnitude and opposite directions at the sensing coil 41 and the offset coil 42, respectively, and the processor can offset a signal for a mixed magnetic field and extract only magnetic signals that are generated magnetic particles by summing up signals acquired at the sensing coil 41 and the offset coil 42. Next, the processor can detect the position of magnetic particles on the basis of the extracted magnetic signal.

Meanwhile, in order to further increase the reception sensitivity for an FOV, the sensing coil 41 may be provided under an FOV and the core center thereof may be disposed over the center of the pair of selection coils 10. In this case, in order to collect magnetic signals throughout the entire are of the FOV, as shown in FIG. 2 and FIG. 9, the area in an x-y plane of the sensing coil 41 may include the area in the x-y plane of the FOV.

The offset coil 42 may be disposed adjacent to the sensing coil 41 in the y-axial direction. In detail, when the outer circumferential surface of at least one of the offset coil 42 and the sensing coil 41 is insulated, the offset coil 42 may be disposed as close to the sensing coil 41 as possible, that is, in an embodiment, the outer circumferential surface of the offset coil 42 may be disposed in contact with the circumferential surface of the sensing coil 41.

According to the structure described above, the position of an FFL in an FOV can be controlled in the x-axial direction and the z-axial direction, and the single side magnetic particle imaging device 1 is pivoted on the FOV in the x-y plane, whereby the FFL can be 3-dimensionally moved.

In this case, the processor can scan magnetic signals generated by magnetic particles through the pair of receiver coils 40. In detail, the processor can be connected to the pair of receiver coils 40 and can recognize only magnetic signals by summing up signals received at the sensing coil 41 and the offset coil 42. The processor can detect the position of magnetic particles by 3-dimensionally scanning magnetic signal by controlling movement of an FFL.

For this operation, the processor may include at least one physical element of application specific integrated circuits (ASICs), digital signal processors (DSPs), digital signal processing devices (DSPDs), programmable logic devices (PLDs), field programmable gate arrays (FPGAs), a controller, and a micro-controller.

As described above, the single side magnetic particle imaging device 1 of the present disclosure is a small-sized coil-based device and has the advantage that it can image an object while being carried anywhere and can secure a wide Field of View (FOV) in comparison to permanent magnet-based devices by imaging magnetic particles through an FFL that is formed in a single direction.

Although the present disclosure was described with reference to the exemplary drawings, it is apparent that the present disclosure is not limited to the embodiments and drawings in the specification and may be modified in various ways by those skilled in the art within the range of the spirit of the present disclosure. Further, even though the operation effects according to the configuration of the present disclosure were not clearly described with the above description of embodiments of the present disclosure, it is apparent that effects that can be expected from the configuration should be also admitted.

What is claimed is:

1. A single side magnetic particle imaging device comprising:
a pair of selection coils arranged in parallel in an x-axial direction and configured to create a y-axial Field Free Line (FFL) in a Field of View (FOV);
a driver coil disposed under the pair of selection coils and configured to move the FFL in a z-axial direction by generating a magnetic field;
An excitation coil disposed over the pair of selection coils and configured to excite magnetic fields in the FOV by generating a magnetic field in the FOV; and
a pair of receiver coils arranged in parallel in a y-axial direction over the excitation coil and configured to receive signals generated by the magnetic particles, respectively.

2. The single side magnetic particle imaging device of claim 1, wherein the pair of selection coils are elliptical flat coils of which a major axis is parallel with the y-axial direction.

3. The single side magnetic particle imaging device of claim 1, wherein currents of the same direction are applied to the pair of selection coils.

4. The single side magnetic particle imaging device of claim 1, wherein the FFL is moved in an x-axial direction in accordance with difference in intensity of currents flowing in two selection coils.

5. The single side magnetic particle imaging device of claim 1, wherein the driver coil is an elliptical flat coil of which a core center is positioned under a center of the pair of selection coils.

6. The single side magnetic particle imaging device of claim 1, wherein the FFL is moved in the z-axial direction in accordance with intensity of a magnetic field generated at the driver coil.

7. The single side magnetic particle imaging device of claim 1, wherein the excitation coil excites the magnetic particles by mixing a high-frequency magnetic field with a magnetic field generated in the FOV.

8. The single side magnetic particle imaging device of claim 1, wherein a frequency of a magnetic field generated at the excitation coil is higher than a frequency of a magnetic field generated at the driver coil, and intensity of the magnetic field generated at the excitation coil is much lower than intensity of the magnetic field generated at the driver coil.

9. The single side magnetic particle imaging device of claim 1, wherein the excitation coil is an elliptical flat coil of which a core center is positioned over a center of the pair of selection coils.

10. The single side magnetic particle imaging device of claim 1, wherein the pair of receiver coils are connected to each other and are wound in opposite directions to each other.

11. The single side magnetic particle imaging device of claim 1, wherein the pair of receiver coils includes:
a circular sensing coil provided under the FOV and having a core center disposed over a center of the pair of selection coils; and
an offset coil disposed adjacent to the sensing coil in the y-axial direction.

12. The single side magnetic particle imaging device of claim 11, wherein an area of the FOV in an x-y plane is included in an area of the sensing coil in the x-y plane.

13. The single side magnetic particle imaging device of claim 1, further comprising a processor configured to detect the position of the magnetic particles by summing up signals acquired at the pair of receiver coils.
